(19) United States
(12) Patent Application Publication
(10) Pub. No.: US 2018/0279920 A1
(43) Pub. Date: Oct. 4, 2018

(54) Device for tracking, monitoring and analyzing Biophysical-activities of users

(71) Applicant: CU Labs, Inc., Mountain View, CA (US)

(72) Inventors: Anal PATEL, Mountain View, CA (US); Susanto PURNAMA, Mountain View, CA (US); Keyur PATEL, Mountain View, CA (US)

(21) Appl. No.: 15/943,918

(22) Filed: Apr. 3, 2018

Related U.S. Application Data
(60) Provisional application No. 62/480,669, filed on Apr. 3, 2017.

(51) Int. Cl. A61B 5/11; A61B 5/00; A63B 24/00

(52) U.S. Cl. CPC A61B 5/1118; A61B 5/6802; A63B 24/0062; A61B 5/7495; A61B 5/749

(57) ABSTRACT

The present disclosure envisages a device for tracking, monitoring and analyzing biophysical-activities of a user. The device includes a motion and activity sensing unit to sense and track motion, direction and acceleration of the biophysical-activities and generate a plurality of motion and activity related signals, a biosensor unit to sense and track a plurality of health parameters and generate a plurality of health related signals, a bio-mechanical tracking unit to sense a plurality of activity parameters including speed, count, axis, direction and position, and generate a plurality of bio-mechanical related signals, a signal conditioning unit to generate conditioned health data and conditioned bio-mechanical data based on the plurality of motion and activity related signals.

### PRIORITY INFORMATION

This application claims priority to U.S. Provisional Application No. 62/480,669 filed on Apr. 3, 2017, the contents of which are hereby incorporated by reference in their entirety.

### TECHNICAL FIELD

The present disclosure relates to devices for monitoring and analysis of health, fitness, and performance of users.

### BACKGROUND

The body weight of an individual is a general indication of an individual's health. To this end, devices like body weighing scales are normally used to measure one's body weight. However, body weight alone is insufficient to allow the individual to gauge his/her fitness and performance. A measure of body fat and biophysical-activity level provides a better assessment of the individual's fitness and also helps the individual to track his/her progress towards achieving fitness and performance goals. Biophysical-activities of a user can be defined as limb movements and body functions during skipping, dancing, free-play, aerobics, rhythmic gymnastics, tai-chi, eurythmy, or in any body flow motion, which are characterized on the basis of grace, rhythm, flow, strength and synchrony.

Conventionally, a number of wearable accessories have been developed in the art which are configured to be worn as bracelets, anklets, and the like. These accessories have sensors which track the heart rate, blood pressure, and other related parameters associated with the fitness of a user. While these accessories do provide measurement of the aforementioned parameters, they are only effective for fitness tracking when the user is particularly involved in fitness activities like jogging, weight training, and the like. These accessories however fail to provide user with monitoring of health/biorhythms or tracking of the movement of body parts, especially the hands, when the user is involved in fitness and performance activities like dancing, aerobics, rhythmic gymnastics, swimming etc. Further, consistently monitoring and recording measured information pertaining to these parameters may be difficult and time consuming for users, especially when the users are using manual means to record relevant data.

Hence, there is felt a need to develop a smart device, for tracking, monitoring and analyzing biophysical-activities of a user, which largely eliminates the aforementioned drawbacks and facilitate health, performance and monitoring.

### OBJECTS

Some of the objects of the present disclosure are aimed to ameliorate one or more problems of the prior art or to at least provide a useful alternative and are listed herein below.

An object of the present disclosure is to provide a device for tracking, monitoring and analyzing biophysical-activities of a user.

Another object of the present disclosure is to provide a device that facilitates real-time analysis and data transmission of fitness, performance activities and human health parameters.

Yet another object of the present disclosure is to provide a device that integrates the signals from a plurality of sensors.

Yet another object of the present disclosure is to provide a device that is easy to use and can be attached to any wearable accessory.

Still another object of the present disclosure is to provide a device which measures and represents accurate results with respect to health, fitness and performance parameters.

One another object of the present disclosure is to provide a device that processes, produces, transmits and represents data with three unique graphic views.

Other objects and advantages of the present disclosure will be more apparent from the following description when read in conjunction with the accompanying figures, which are not intended to limit the scope of the present disclosure.

### SUMMARY

The present disclosure envisages a device for tracking, monitoring and analyzing biophysical-activities of a user, the device comprising a processing unit, a motion and activity sensing unit, a biosensor unit, a bio-mechanical tracking unit and a signal conditioning unit.

The processing unit includes at least one processor. The processing unit is configured to receive predetermined set of rules from a rules repository and is further configured to generate processing commands sequentially.

The motion and activity sensing unit is configured to sense and track motion, direction and acceleration of the biophysical-activities performed by the user and is further configured to generate a plurality of motion and activity related signals.

The biosensor unit is configured to sense and track a plurality of health parameters of the user while performing the biophysical-activities and is further configured to generate a plurality of health related signals.

The bio-mechanical tracking unit is configured to sense a plurality of activity parameters including speed, count, axis, direction and position, and is further configured to generate a plurality of bio-mechanical related signals based on the sensed activity parameters.

The signal conditioning unit co-operates with the motion and activity sensing unit, the biosensor unit and the bio-mechanical tracking unit to receive the plurality of motion and activity related signals, the plurality of health related signals and the plurality of bio-mechanical related signals respectively. The signal conditioning unit is configured to generate conditioned health data and conditioned bio-mechanical data based on the plurality of motion and activity related signals.

In an embodiment, the device is communicatively coupled to a remote unit. The remote unit is configured to receive and display the conditioned health data and conditioned bio-mechanical data of the performed biophysical-activity of the user.

In an embodiment, the motion and activity sensing unit comprises an accelerometer and a gyroscope configured to sense and track the motion, direction and acceleration of the biophysical-activities performed by the user.

In an embodiment, the biosensor unit comprises a plurality of sensors integrated with each other, configured to sense and track health parameters of the user while performing the biophysical-activity.

In an embodiment, the processing unit comprises a data repository. The data repository is configured to store and periodically update the conditioned health data and conditioned bio-mechanical data of biophysical-activities performed by the user.

In an embodiment, the device includes a gesture control unit. The gesture control unit is configured to detect gestures of the user while performing the biophysical-activities and correspondingly generate a plurality of gesture signals.

In an embodiment, the gesture control unit is adapted to cooperate with the signal conditioning unit to receive the plurality of gesture signals and generate conditioned gesture data.

In an embodiment, the gesture control unit includes at least one proximity sensor.

In an embodiment, the processing unit further comprises a wireless transceiver unit and a memory. The wireless transceiver unit is configured to receive and transmit the conditioned health data and the conditioned bio-mechanical data associated with the user. The memory is configured to store a pre-determined set of threshold ranges of health and bio-mechanical data associated with the user.

In an embodiment, the processor is selected from the group consisting of an application specific integration circuit (ASIC), an Integrated Circuit based processor, a PIC controller, and combination thereof.

In an embodiment, the device further comprises a power management module. The power management module is configured to regulate the supply and distribution of power to the device.

In an embodiment, the remote unit is a multi-mode notification module with one or more notification/display units.

In an embodiment, the processor includes an audio processor.

In an embodiment, the processor further comprises a microphone.

The microphone is configured to receive verbal instructions from the user and is further configured to, under processing commands, generate, transmit and implement verbal instruction signals.

In an embodiment, the device further comprises a speaker for playing music or for providing notifications or instruction cues with respect to the biophysical-activities performed by the user.

In an embodiment, the device further comprises a connectivity interface. The connectivity interface is configured to facilitate recharging of the device and is further configured to connect the device with electronic appliances.

In an embodiment, the biosensor unit includes a touch ID sensor. The touch ID sensor is configured to facilitate user sign-in by comparing fingerprints of the user with stored fingerprints.

In an embodiment, the data repository is further configured to store fingerprints of the user.

### BRIEF DESCRIPTION OF ACCOMPANYING DRAWING

A device of the present disclosure will now be described with the help of the accompanying drawing, in which:

FIG. 1 illustrates a block diagram depicting the device, in accordance with an embodiment of the present disclosure; and

FIG. 2 illustrates an accessory integrated with the device, in accordance with an exemplary and preferred embodiment of the present disclosure.

### LIST AND DETAILS OF REFERENCE NUMERALS USED IN THE DESCRIPTION AND DRAWING:

| Reference numeral | References associated with reference numeral |
|---|---|
| 100 | Device |
| 105 | Processor |
| 110 | Rules Repository |
| 115 | Data Repository |
| 120 | Motion and Activity Sensing Unit |
| 125 | Biosensor unit |
| 130 | Bio-mechanical Tracking Unit |
| 135 | Signal Conditioning Unit |
| 140 | Gesture Control Unit |
| 150 | Remote unit |
| 200 | Accessory |
| 250 | A plurality of sensors |

### DETAILED DESCRIPTION

The body weight of an individual is a general indication of an individual's health. To this end, devices like body weighing scales are normally used to measure one's body weight. However, body weight alone is insufficient to allow the individual to gauge his/her fitness and performance. A measure of body fat and biophysical-activity level provides a better assessment of the individual's fitness and also helps the individual to track his/her progress towards achieving fitness and performance goals. Biophysical-activities of a user can be defined as limb movements and body functions during, dancing, free-play, aerobics, rhythmic gymnastics, tai-chi, eurythmy, or in any body flow motion, which are characterized on the basis of grace, rhythm, flow, strength and synchrony.

Conventionally, a number of wearable accessories have been developed in the art which are configured to be worn as bracelets, anklets, and the like. These accessories have sensors which track the heart rate, blood pressure, and other related parameters associated with the fitness of a user. While these accessories do provide measurement of the aforementioned parameters, they are only effective for fitness tracking when the user is particularly involved in fitness activities like jogging, weight training, and the like. These accessories however fail to provide user with monitoring of health/biorhythms or tracking of the movement of body parts, especially the hands, when the user is involved in fitness and performance activities like dancing, aerobics, rhythmic gymnastics, swimming, etc. Further, consistently monitoring and recording measured information pertaining to these parameters may be difficult and time consuming for users, especially when the users are using manual means to record relevant data.

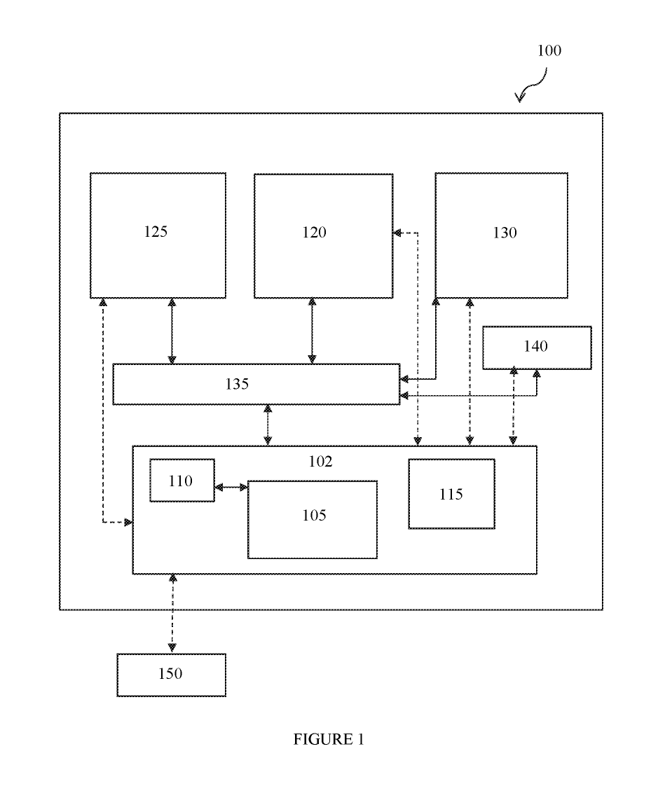

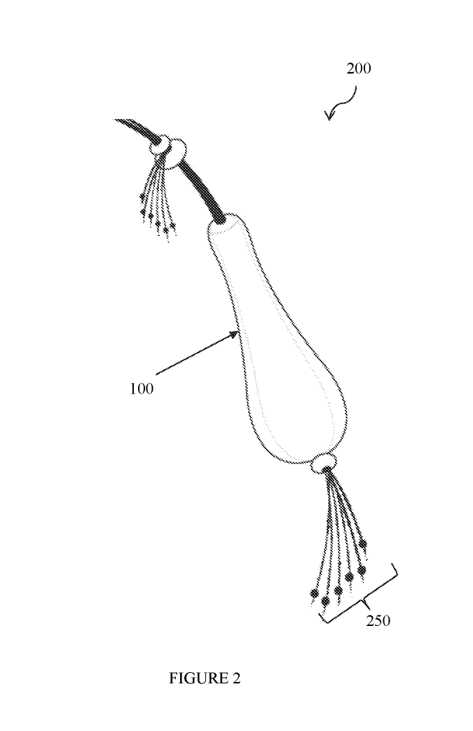

In subsequent paragraphs, the present disclosure envisages a device for tracking, monitoring and analyzing biophysical-activities of a user, which alleviates the aforementioned drawbacks. The device, in accordance with an embodiment of the present disclosure will now be described with reference to the embodiments, which do not limit the scope and ambit of the disclosure. The description of the device is provided purely by way of example and illustration. The present disclosure has been described herein below with reference to FIG. 1 and FIG. 2. FIG. 1 illustrates a block diagram depicting a device 100, in accordance with an embodiment of the present disclosure. FIG. 2 illustrates an accessory 200 in the form of a skipping rope, integrated with a device 100, in accordance with an exemplary and preferred embodiment of the present disclosure. The solid lines in the FIG. 1 indicate transmission of signals/processing commands and the dotted lines indicate transfer of data/information.

The present disclosure discloses a device 100 for tracking, monitoring and analyzing biophysical-activities performed by users. Typically, the device 100 is a fitness and performance measurement device which measures real-time motion, direction and acceleration data generated by any biophysical-activity performed by the user including, but not limited to, limb movements during dancing, free-play, aerobics, rhythmic gymnastics, tai-chi, eurythmy, or any other body flow motion, and thereby reducing or eliminating any human intervention during tracking, monitoring and analyzing of such biophysical-activities performed by users. The device 100 is configured to track, monitor and analyze fitness and the performance of a user on the basis of grace, rhythm, flow, strength and synchrony.

The working of the device 100 is described herein with reference to FIG. 1, in accordance with an embodiment of the present disclosure.

The device 100 may comprise a processing unit 102, a motion and activity sensing unit 120, a biosensor unit 125, a bio-mechanical tracking unit 130, and a signal conditioning unit 135. The fitness and performance measurement device 100 may also involve a remote unit 150 which is configured to be communicatively coupled to the device 100. In an embodiment, the device 100 is part of or attached to an accessory 200 as shown in FIG. 2. The accessory can be a rope, and wearable accessories like tie, ribbons, scarves, garments or any other custom artistic expression to which the device 100 is attached.

In a preferred embodiment, the device 100 requires a user to hold the device 100 in his/her hand or wear the accessory 200 to which the device 100 is attached. The housing of the device 100 is typically of a polymeric material such as poly-carbonate or natural or synthetic wood such as PlanWood™. In an embodiment, the device 100 is rubber coated for better gripping. In an embodiment, the device 100 is provided with water/heat transfer print finish. In an embodiment, some portion of the device 100 may be made of other material such as metal, cotton, and wool.

The processor 105 of the device 100 is configured to receive pre-determined set of rules from a rules repository 110 and is further configured to generate processing commands sequentially that guides the functioning of the device 100. For example, the rules repository 110 may have rules as per which the processes of the device 100 will work.

In one embodiment, the device 100 is in the form of a handle which can be held by a user performing biophysical-activities. In a preferred embodiment, the device 100 has built in it the processing unit 102 having at least one processor 105, the rules repository 110, and a data repository 115.

In an embodiment, the data repository 115 of the processing unit 102 is configured to store predetermined conditioned health data and conditioned bio-mechanical data of biophysical-activities of a user. In another embodiment, the device 100 may further comprise an updater, which under processing commands generated by the processor 105, updates or edits data stored in the data repository 115 in accordance with data received from the device 100 during or after the user performs a biophysical-activity, initially in the training phase and subsequently in the dynamic learning phase of the device 100. The initial training phase and the dynamic learning phase of the device ensure that the device 100 is continuously improving organically.

The motion and activity sensing unit 120 of the device 100 is configured to sense and track motion, direction and acceleration of the biophysical-activities performed by a user in real-time. The motion and activity sensing unit 120 is further configured to generate a plurality of motion and activity signals based on the sensed and tracked motion, direction and acceleration. In an embodiment, the device 100 comprises an accelerometer and a gyroscope configured to sense and track motion, direction and acceleration of the biophysical-activities performed by a user respectively. The motion and activity sensing unit 120 tracks, monitors and analyzes real-time motions activity and intensity on the basis of a predefined algorithm.

In an embodiment, the motion and activity sensing unit 120 can also facilitate 3D motion tracking and later may communicate with the processing unit 102 and the remote unit 150 to display such tracking and analysis in the form of graphical representations. In another embodiment, the motion and activity sensing unit 120 is configured to provide video and overlay performance data graphing. In yet another embodiment, the motion and activity sensing unit 120 also assists in the learning of the device 100 so as to make recommendations for fitness and performance parameters such as motion, pace, rhythm, speed, synchrony, routine and the like.

The biosensor unit 125 of the device 100 is configured to sense and track a plurality of health parameters of the user while performing the biophysical-activities. The biosensor unit 125 is further configured to generate a plurality of health related signals based on the sensed and tracked plurality of health parameters. In an embodiment, the biosensor unit 125 comprises a plurality of sensors 250 to sense and track health parameters including, but not limited to, heart rate and pulse rate, heart and pulse rhythm, variability in heart and pulse rhythm, heart rate and pulse rate, perspiration level for hydration/dehydration, body temperature, SPO2 (peripheral capillary oxygen saturation level), VO2 (Maximum Oxygen Consumption), blood pressure, blood glucose level, galvanic skin response, respiration rate, UV exposure and serum Vitamin D level, blood lactate, hormone levels in perspiration (sweat) and the like, of the user while performing the biophysical-activity. In an embodiment, the biosensor unit 125 includes a touch ID sensor. The touch ID sensor is configured to facilitate user(s) sign-in by comparing the fingerprint of the user(s) with stored fingerprints of the user(s). In an embodiment, the fingerprints of the user are stored in the data repository 115. The touch ID sensor helps in configuring the device (100) based on the identified user.

In an embodiment, the plurality of sensors 250 is externally mounted on the device 100 by means of a bead placed at the bottom of the device and tassels.

The bio-mechanical tracking unit 130 of the device 100 may be configured to sense and track a plurality of activity related parameters including speed, count, acceleration, axis, and position, and is further configured to generate a plurality of bio-mechanical signals based on the sensed and tracked plurality of activity parameters.

The signal conditioning unit 135 of the device 100 is configured to co-operate with the motion and activity sensing unit 120, the biosensor unit 125, and the bio-mechanical tracking unit 130 to receive a plurality of motion and activity related signals, a plurality of health related signals and a plurality of bio-mechanical related signals respectively. The signal conditioning unit 135 is further configured to, under processing commands, generate conditioned health data and conditioned bio-mechanical data based on the plurality of motion and activity related signals.

In an embodiment, the generated conditioned health data and conditioned bio-mechanical data of the user while performing the biophysical-activities is stored and periodically updated in the data repository 115 of the device 100. In another embodiment, the device 100 further comprises a health parametric learning engine that can continuously analyze the health parameters of a user and keep the device 100 updated with the user's health data.

The remote unit 150 is communicatively coupled to the device 100. In an embodiment of the present disclosure, the remote unit 150 is configured to receive, analyze and display conditioned health data and conditioned bio-mechanical data of the performed biophysical-activity to a user by means of a display unit.

The device 100 may further include a gesture control unit 140 which is configured to detect gestures of the user while performing the biophysical-activities and generate a plurality of gesture related signals based on the detected gestures. In an embodiment, the gesture control unit 140 is adapted to cooperate with the signal conditioning unit 135 such that the signal conditioning unit 135 receives the plurality of gesture related signals and generates conditioned gesture data. In another embodiment, the gesture control unit 140 includes at least one proximity sensor.

In an embodiment of the present disclosure, the processing unit 102 of the fitness and performance measurement device 100 further comprises: (i) a wireless transceiver unit which is configured to receive and transmit conditioned health data and conditioned bio-mechanical data related to a user, and (ii) a memory which is configured to store a pre-determined set of threshold ranges of health and bio-mechanical data related to a user.

In yet another embodiment, the at least one processor 105 of the processing unit 102 is selected from the group consisting of an application specific integration circuit (ASIC), Integrated Circuit based processor, a PIC controller, and combination thereof.

The fitness and performance measurement device 100 of the present disclosure may further comprise a power management module which can be configured to regulate the supply and distribution of power to run the fitness and performance measurement device 100.

In an embodiment, the remote unit 150 is a multi-mode notification module with one or more notification/display units.

In another embodiment, the at least one processor 105 includes an audio processor.

In still another embodiment, the fitness and performance measurement device 100 may further comprise a microphone which is configured to receive verbal instructions from a user and may be further configured to, under processing commands, generate, transmit and implement verbal instruction signals.

In still another embodiment, the fitness and performance measurement device 100 may further comprise a speaker for playing music or for providing notifications or instruction cues with respect to the biophysical-activities performed by the user.

Further, the fitness and performance measurement device 100 may comprise a connectivity interface to facilitate recharging of the device 100 and is further configured to connect the device 100 with electronic appliances.

### Technical Advancements and Economical Significance

A device for tracking, monitoring and analyzing biophysical-activities of users in accordance with the present disclosure described herein above has several technical and economic advantages including but not limited to providing a device that:
provides real-time analysis and data transmission of fitness and performance activities and human health parameters;
integrates signals from a plurality of sensors;
is easy to use and can be attached to any wearable accessory;
measures and represents accurate results with respect to health, fitness and performance parameters; and
processes, produces, transmits and represents data with three unique graphic views.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or mixtures or quantities, as the use may be in the embodiment of the disclosure to achieve one or more of the desired objects or results.

Any discussion of documents, acts, materials, devices, accessories or the like that has been included in this specification is solely for the purpose of providing a context for the disclosure. It is not to be taken as an admission that any or all of these matters form part of the prior art base or were common general knowledge in the field relevant to the disclosure, as it existed anywhere before the priority date of this application.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

1. A device (100) for tracking, monitoring and analyzing biophysical-activities of a user, said device (100) comprising:
a processing unit (102) having at least one processor (105) configured to receive predetermined set of rules from a rules repository (110) and further configured to generate processing commands sequentially;
a motion and activity sensing unit (120) configured to sense and track motion, direction and acceleration of the biophysical-activities performed by the user and further configured to generate a plurality of motion and activity related signals;
a biosensor unit (125) configured to sense and track a plurality of health parameters of the user while performing the biophysical-activities and further configured to generate a plurality of health related signals;
a bio-mechanical tracking unit (130) configured to sense a plurality of activity parameters including speed, count, axis, direction and position, and further configured to generate a plurality of bio-mechanical related signals based on said sensed activity parameters; and
a signal conditioning unit (135) cooperates with said motion and activity sensing unit (120), said biosensor unit (125), and said bio-mechanical tracking unit (130) to receive said plurality of motion and activity related signals, said plurality of health related signals and said plurality of bio-mechanical related signals respectively,
characterized in that said signal conditioning unit (135) is configured to, under the processing commands, generates conditioned health data and conditioned bio-mechanical data based on said plurality of motion and activity related signals.

2. The device (100) as claimed in claim 1, wherein said device (100) is communicatively coupled to a remote unit (150), said remote unit (150) configured to receive and display said conditioned health data and conditioned bio-mechanical data of the performed biophysical-activity of the user.

3. The device (100) as claimed in claim 1, wherein said motion and activity sensing unit (120) comprises an accelerometer and a gyroscope configured to sense and track the motion, direction and acceleration of the biophysical-activities performed by said user.

4. The device (100) as claimed in claim 1, wherein said biosensor unit (125) comprises a plurality of sensors (250) integrated with each other, configured to sense and track health parameters of said user while performing the biophysical-activity.

5. The device (100) as claimed in claim 1, wherein said processing unit (102) further comprises a data repository (115) configured to store and periodically update said conditioned health data and conditioned bio-mechanical data of biophysical-activities performed by the user.

6. The device (100) as claimed in claim 1, which includes a gesture control unit (140), configured to detect gestures of the user while performing the biophysical-activities and correspondingly generate a plurality of gesture signals.

7. The device (100) as claimed in claim 6, wherein said gesture control unit (140) is adapted to cooperate with said signal conditioning unit (135) to receive said plurality of gesture signals and generate conditioned gesture data.

8. The device (100) as claimed in claim 6, wherein said gesture control unit (140) includes at least one proximity sensor.

9. The device (100) as claimed in claim 1, wherein said processing unit (102) further comprises:
a wireless transceiver unit configured to receive and transmit said conditioned health data and said conditioned bio-mechanical data associated with the user; and
a memory configured to store a pre-determined set of threshold ranges of health and bio-mechanical data associated with the user.

10. The device (100) as claimed in claim 1, wherein said at least one processor (105) is selected from the group consisting of an application specific integration circuit (ASIC), an Integrated Circuit based processor, a PIC controller, and combination thereof.

11. The device (100) as claimed in claim 1, which further comprises a power management module configured to regulate the supply and distribution of power to said device (100).

12. The device (100) as claimed in claim 2, wherein said remote unit is a multi-mode notification module with one or more notification/display units.

13. The device (100) as claimed in claim 1, wherein said at least one processor (105) includes an audio processor.

14. The device (100) as claimed in claim 1, which further comprises a microphone configured to receive verbal instructions from the user and further configured to, under the processing commands, generate, transmit and implement verbal instruction signals.

15. The device (100) as claimed in claim 1, which further comprises a speaker for playing music or for providing notifications or instruction cues with respect to the biophysical-activities performed by the user.

16. The device (100) as claimed in claim 1, which further comprises a connectivity interface configured to facilitate recharging of said device (100) and further configured to connect said device (100) with electronic appliances.

17. The device (100) as claimed in claim 1, wherein the biosensor unit (125) includes a touch ID sensor configured to facilitate user sign-in by comparing fingerprints of the user with stored fingerprints.

18. The device (100) as claimed in claim 17, wherein the data repository (115) is further configured to store fingerprints of the user.
